SEQUENCE LISTING (2 sequences)

SEQ ID NO: 1 - DNA, 19 bp, Artificial Sequence
  PCR Primer
cagatgcarcgattcagtg

SEQ ID NO: 2 - DNA, 24 bp, Artificial Sequence
  PCR Primer
agggcattttggacaaagcgtcta

(19) United States
(12) Patent Application Publication
(10) Pub. No.: US 2011/0002959 A1
(43) Pub. Date: Jan. 6, 2011

(54) Vaccine Production For Pathogenic Bird Viral Diseases

(75) Inventors: James K. Petell, Grand Forks, ND (US); James M. Schiltz, Sisseton, SD (US); David S. Bradley, Grand Forks, ND (US); Barry I. Milavetz, Grand Forks, ND (US); Lata Balakrishnan, Rochester, NY (US)

Correspondence Address: WOMBLE CARLYLE SANDRIDGE & RICE, PLLC, ATTN: IP DOCKETING, P.O. BOX 7037, ATLANTA, GA, 30357-0037, US

(73) Assignee: Avianax, LLC

(21) Appl. No.: 12/827,052

(22) Filed: Jun. 30, 2010

Related U.S. Application Data
(62) Division of application No. 12/164,940, filed on Jun. 30, 2008, now abandoned.
(60) Provisional application No. 60/937,653, filed on Jun. 29, 2007.

(51) Int. Cl. A61K 39/12; A61P 31/12; C12N 7/02

(52) U.S. Cl. 424/204.1; 435/239

(57) ABSTRACT

The present invention is an improved method for the production of vaccines to transmittable viral pathogens where the virus is pathogenic to the chicken embryos. Bird embryos are selected for vaccine production from wild and domestic birds, and preferably waterfowl, that have increased resistant to the viral pathogen. The invention is useful for native and engineered viruses.

### CROSS-REFERENCE TO RELATED APPLICATIONS

The present patent application is a divisional of U.S. patent application Ser. No. 12/164,940, filed Jun. 30, 2008, which claims priority to U.S. Provisional Patent Application No. 60/937,653, filed Jun. 29, 2007, both of which are incorporated herein by reference in their entirety.

### FIELD OF THE INVENTION

The present invention relates to vaccines for viral infection and methods of preparation of such vaccines. The invention also relates to compositions and methods of preparation of therapeutic treatments for various viral agents.

### BACKGROUND OF THE INVENTION

The H5N1 strain of Avian Influenza (AI), also known as “Bird Flu” or highly pathogenic avian influenza (HPAI), is expected to hit pandemic proportions worldwide in the near future. The mortality rate of 30% to 70% in known infected patients (as reported by the Centers for Disease Control) makes it one of the deadliest viruses since the Spanish flu of 1918 when the wrong population was treated due to limited supplies and over 500,000 people died alone in the United States.

One of the major drawbacks in the manufacture of a vaccine to prevent H5N1 is the fact that this particular virus kills not only the domesticated chickens but also chicken embryos. In order to make the vaccine production feasible in chicken eggs, the 1997 strain of H5NI virus was reverse engineered over the course of five years to make it less lethal to chicken embryos. Although this appears to be effective in producing the vaccines, it suffers from two limitations. First, the amount of vaccine or doses produced per egg appears to be substantially decreased relative to the common annual flu virus. This means that the typical 100,000,000 eggs used to produce flu vaccine corresponding to 185 million doses will only be able to vaccinate a small part of the U.S. population. Second, the strain is a 1997 isolate and potentially the additional mutation may render the vaccine less effective to the current strain.

The other method to produce a “recombinant DNA” vaccine encompasses the use of cell culture. Recently, Gambotto et al at the University of Pittsburgh reported that mice were protected against a 2004 strain H5N1 when injected twice with an engineered adenovirus containing several portions hemagglutinin (HA) gene derived from the same strain. Although their work in mice and companion work in chickens shows potential, in a recent review by Cui et al (Advances in Genetics: 54, 2005) there are a number of considerations that suggest that this work will not translate into a viable human vaccine in the near future. First, the 15 year history of active research in the field of DNA vaccines demonstrates that, even with promising findings in mice or other laboratory rodents, the progression to appropriate immune responses in primates, human, and others remains elusive. The vast majority of human trials, primarily for HIV and malaria, remain in the safety phase, with only partial protection shown in the few that have proceeded beyond this phase. One major problem is that the viral or bacterial DNA vaccines are relatively poor immunogens (i.e., they do not induce a strong response against the desired microorganism). Others have attempted to overcome this by either increasing the dosage of vaccine or by the addition of immunostimulatory molecules. The first solution works well in mice but is difficult to achieve when translating work to humans due to the volume necessary and the time and cost necessary to produce the number of vaccine units. Another significant hurdle is that although DNA vaccines may produce an effective response in small animal models, they have produced weak antibody responses in humans. Thus, even if helpful for ongoing disease, these will not provide protection from new infections, a paramount requirement of any vaccine. Moreover, the U.S. production capacity of large doses of vaccine using cell culture is not currently available and may take several years to achieve. While solutions to these problems are being sought, the potential for mutation of H5N1 may result in a vaccine with reduced efficacy prior to achieving mass production.

Therefore, there remains an unmet need for vaccine production against avian influenza or other viruses where the virus is deleterious to the host chicken embryos and therefore eliminates or reduces vaccine production. In addition, the vaccine produced must be immunogenic, scalable for large production, and cost effective.

### SUMMARY OF THE INVENTION

The invention herein relates to vaccines and therapeutic treatments for transmittable viral pathogens. In particular embodiments, the invention provides compositions and methods of preparation thereof that are advantageous for the improved production of vaccines in eggs, particularly the production of influenza vaccines. The inventive methods overcome limitations in the art where virus production is reduced using traditional passage methods in chicken eggs due to increased pathogenicity to chicken embryos. The present invention, however, has realized the ability to easily and effectively prepare vaccines by other methods. The invention also provides therapeutic compositions and methods of preparation thereof that are advantageous for the improved production of treatments of viral diseases, particularly influenza treatments.

As used herein, the word “pathogenic” means the causing by a biological agent of a disease or illness to its host. As used herein, the word “resistance” means reduced incidence in a bird species of disease or illness to a pathogen relative to incidence of disease or illness in another bird species.

In one aspect, the invention is directed to a method for producing a vaccine against a transmittable virus that is pathogenic to a bird species susceptible to the virus. In one embodiment, the method comprises: a) selecting an embryo of a second bird species that is different from the susceptible bird species and that exhibits resistance to the transmittable virus; b) injecting into the embryo an amount of the transmittable virus; c) incubating the embryo for a period of time after injection effective for virus production in the embryo; and d) removing fluid from the embryo containing the produced virus.

In some embodiments, the method can further comprise inactivating the virus. Such inactivation can be carried out in ovo (i.e., while the embryo is still in the egg), or can be carried out after removal from the embryo.

In other embodiments, the method can further comprise injecting the vaccine into an animal for protection of the animal against said transmittable virus. Such animals can include birds (e.g., geese, ducks, turkeys, pigeons, ostriches, and chickens) and mammals (e.g., goats, horses, rabbits, rats, mice, pigs, cows, and humans).

The method can be characterized by the injection site of the virus. For example, the transmittable virus may be injected in a specified egg compartment. In certain embodiments, the egg compartment is selected from a group consisting of the air sac, the allantoic cavity, and combinations thereof.

The transmittable viral disease can be selected from a variety of viruses and can be a native virus or an engineered virus. Non-limiting examples of a transmittable virus that may be used in the preparation of a vaccine thereto include West Nile Virus, Hepatitis virus, HIV, RSV, CMV, HSV, ESV, VSV, viral encephalitide, viral hemorrhagic fever, avian influenza virus, and combinations thereof. Specific, non-limiting examples of viral encephalitides include Eastern equine encephalomyelitis virus, Venezuelan equine encephalomyelitis virus, Western equine encephalomyelitis virus, and combinations thereof. Specific, non-limiting examples of avian influenza viruses include H5N1, H5N2, H5N8, H5N9, H7N1, H7N3, H7N4, H7N7, H9N2, and combinations thereof.

In one embodiment, the invention also comprises a composition for use in a vaccine against a transmittable virus that is pathogenic to a chicken embryo. In specific embodiments, the composition comprises virus particles obtained from a bird embryo of a bird species that exhibits resistance to the transmittable virus. Moreover, the composition may specifically be useful in a vaccine against an avian influenza virus, and the composition may particularly comprise virus particles obtained from a goose embryo.

In another aspect, the invention provides methods of forming a composition comprising antibodies useful in the therapeutic treatment of a viral disease. In some embodiments, the method comprises: a) injecting a live goose with an agent comprising or derived from the viral disease; b) providing an incubation period wherein the goose develops antibodies to the viral disease; c) retrieving an egg laid by the injected goose, the egg comprising antibodies to the viral disease; and d) obtaining from the egg antibodies to the viral disease.

The method may further comprise combining the antibodies with a pharmaceutically acceptable carrier. In specific embodiments, the viral disease is an avian influenza virus.

The invention also comprises a composition for therapeutic treatment of a viral disease prepared according to the above method.

### BRIEF DESCRIPTION OF THE DRAWINGS

Having thus described the invention in general terms, reference will now be made to the accompanying drawings, wherein:

FIG. 1 is an RT-PCR analysis of goose embryo allantoic fluid for avian influenza virus RNA after virus infection;

FIG. 2 is an RT-PCR product from RNA extract of H1N1 AI-infected goose eggs; and

FIG. 3 is a chart illustrating the resistance of goose eggs vs. turkey eggs to H1N1 AI Infection.

### DETAILED DESCRIPTION OF INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used herein is for describing particular embodiments only, and is not intended to be limiting. As used herein, the singular forms “a,” “an,” and “the” include plural referents unless the context clearly dictates otherwise.

The current strain of highly pathogenic avian influenza, H5N1, exhibits very high mortality in chickens and turkeys, approaching or achieving 100% mortality. Wild birds are recognized as potential carriers of the H5N1 strain, however the mortality rates of wild birds infected with H5N1 remains largely unknown. Recent studies have shown various H5N1 strain variants cause substantially reduced or no mortality in domestic waterfowl relative that observed in chickens. In contrast to HPAI, West Nile Virus has been reported to cause mortality in geese but no mortality in chickens and turkeys. However, little or no research has been performed to determine the mortality of bird embryos to the HPAI variants, West Nile Virus, or other avian viruses.

In the past, chicken embryos have been used exclusively in vaccine production because of their large availability, economy, size, freedom from microbial contamination, and lack of residual antibodies against the virus. Typically, chicken embryos at 9 to 11 days of ages are selected for virus production. In vaccine production to the common influenzas, the embryos are injected with stock virus and incubated for an effective time to maximize virus production, generally 1 to 6 days. Vaccine production has occurred with differential success, however in the case of the current HPAI H5N1, there has been an approximate 100% mortality rate in domestic chickens and turkeys as well as embryos. Accordingly, to be able to produce virus in chicken eggs, the virus was genetically reverse engineered to reduce its pathogenicity to chickens. This method, however, results in diminished virus production.

The present invention overcomes these problems by providing methods of producing vaccines to transmittable viral pathogens by passage of virus in a bird embryo that has an increased resistance to the pathogen relative to chicken embryos. The reduced resistance may include reduced mortality.

Accordingly, in certain embodiments, the present invention is directed to a method for the improved production of a vaccine against a transmittable virus that is pathogenic to a bird (i.e., a bird that is susceptible to the virus). More particularly, the method can comprise selecting an embryo of a second bird species that is different from the susceptible bird species and that exhibits resistance to the transmittable virus. The method can further comprise injecting into the embryo an amount of the virus effective to elicit production of an additional amount of the virus, which may further include incubating the embryo for a period of time effective for virus production. Further, the method may comprise removing fluid containing the produced virus.

The method of the invention is useful in the production of a vaccine against a variety of transmittable viruses. For example, the transmittable virus can comprise a native virus (i.e., of natural origin) or can comprise and engineered transmittable virus. In certain embodiments, the transmittable virus cab be selected from the group consisting of West Nile Virus (WNV), Hepatitis virus (including Hepatitis A, Hepatitis B, and Hepatitis C), human immunodeficiency virus (HIV), respiratory syncital virus (RSV), cytomegalovirus (CMV), herpes simplex virus (HSV1 and HSV2), ectocarpus siliculosus virus (ESV), vesicular stomatitis virus (VSV), viral encephalitide, viral hemorrhagic fever, and avian influenza virus. Moreover, combinations of viruses may be used. In specific embodiments, viral encephalitide can be selected from the group consisting of Eastern equine encephalomyelitis virus, Venezuelan equine encephalomyelitis virus, Western equine encephalomyelitis virus, and combinations thereof. In other specific embodiments, avian influenza viruses can be selected from the group consisting of H5N1, H5N2, H5N8, H5N9, H7N1, H7N3, H7N4, H7N7, H9N2, and combinations thereof.

The present invention is particularly useful in that it is possible to prepare vaccines easily and in large quantities where it has heretofore not been possible. As previously pointed out, many viruses, such as HPAI H5N1, are lethal to chickens. Accordingly, it is difficult to prepare vaccines to such viruses by passing the virus through a chicken embryo (i.e., injecting the virus into a chicken egg). Chickens are thus an example of a bird species that is susceptible to HPAI H5N1, since HPAI H5N1 is pathogenic to chickens. The present invention, however, has recognized the ability to easily and effectively produce a vaccine to HPAI H5N1 by passing the virus through the embryo of a bird species that is different from chickens and that exhibits resistance to HPAI H5N1. Further, the invention has realized the ability to easily and effectively produce vaccines to other viruses that are pathogenic to a particular bird species (i.e., the “susceptible bird species”) by passing the virus through the embryo of a bird species that is different from the susceptible bird species and that exhibits resistance to the transmittable virus.

A variety of bird species can be used in the present invention. Generally, the invention can comprise evaluating one or more bird species to establish the pathogenic effect of a specific virus of interest on a particular bird species. When the specific virus is found to be pathogenic to a specific bird species but a second, different bird species exhibits resistance to the virus, the second bird species can be used in carrying out the methods of the invention to prepare a vaccine against the specific virus.

In certain embodiments, it is useful to use embryos of waterfowl bird species for the preparation of a vaccine according to the invention. For example, waterfowl bird species, such as a goose and duck, are more resistant to the H5N1 virus and can be particularly useful for production of higher virus levels in relation to the use of chicken eggs. Both geese and ducks have larger eggs than chickens. The expected volume of a goose egg is approximately 5-10 times the volume of a chicken egg. Importantly, the use of resistant bird eggs (e.g., goose or duck) may not require that the virus be modified before injection into the bird egg. This means that current viral strains could be used as a source of vaccine without the need for genetic modification. Nevertheless, even if an engineered virus is found to be required (or simply more desirable), the production of virus particles in more resistant eggs, according to the present invention, would be expected to result in the production of greater virus volumes as compared to the use of chickens.

A critical consideration in relation to the time of inoculation is the position of the inner egg structure. The two primary regions of virus injection for vaccine production are the air sac and the allantoic fluid cavity. In some embodiments, the virus is injected into the air sac. In other embodiments, the virus is injected into the allantoic cavity. In still other embodiments, the virus is injected into both the air sac and the allantoic cavity.

To carry out the method, egg development should preferably have proceed for a sufficient time such that the amnion is maximally enlarged to facilitate needle penetration while ensuring the maximum possible amount of allantoic fluid is present. In addition, the allantoic region is preferred because of potential maternal antibodies present in the yolk. Determination of the preferred site of injection of the virus in ovo was achieved according to the invention through carrying out a variety of test procedures.

The mechanism of virus injection has been performed in the art by drilling a hole through the shell to provide access for injection. The key aspect of the drilling is to not cause damage to embryo tissues and organs and the extraembyronic membranes surrounding the embryo. An automated system has been described in U.S. Pat. No. 4,040,388, which is incorporated herein by reference and would be suitable for large scale production. The resulting hole may or may not be resealed; however, care should be taken to avoid contamination by bacterial pathogens during the drilling process and before or after injection.

In the event that multiple strains of the transmittable virus are present, two or more strains can be inoculated individually into resistant bird embryos and the allantoic fluids are pooled to provide broader protection to strain variants. For common influenza vaccine, for example, it is typical to use three predominant strains from the past and/or during the present year. After the allantoic fluids are pooled, a number of methods have been used to simplify the recovery of the virus or viral products from the allantoic fluids. Extraction of virus from concentrated allantoic fluid was performed using diethyl ether or methylacetate, and improved processes were obtained using multiple extractions with both butyl and ethyacetates. Such methods are described in U.S. Pat. No. 3,627,873 and U.S. Pat. No. 4,000,527, both of which are incorporated herein by reference in their entirety.

Other methods have utilized a multi-step extraction process that removes virus particles from cellular debris and is useful for chick allantoic fluid. See U.S. Pat. No. 3,316,153, which is incorporated herein by reference in its entirety. The virus particles are precipitated using calcium phosphate and resolubilized using EDTA. Still other methods used ion exchange chromatography, preferably cellulose sulfate column, whereby the allantoic fluid is applied to the column to bind the virus particles, washed with 1.0 M sodium chloride and finally eluted with approximately 5.0 M sodium chloride. See U.S. Pat. No. 4,724,210, which is incorporated herein by reference in its entirety.

Yet other methods to isolate virus particles subjected allantoic fluid to high-speed centrifugation. See U.S. Pat. No. 3,962,421, which is incorporated herein by reference in its entirety. The virus pellet is resuspended in saline and ball-milled for 12-15 hours to make a virus suspension. To produce lipid-free particles containing surface antigens the suspension was treated with phosphate ester.

In order to improve virus yields, recent work has reported that using total non-isotonic salt concentration of 0.5 M or greater in the allantoic fluid was able to increase the virus extraction from cellular debris containing allantoic fluid. See U.S. Patent Application Publication No. 20050186223, which is incorporated herein by reference in its entirety. The preferred pH range is 3.0 to 10.0 in a 20 to 250 mM phosphate buffer.

Surprisingly, the present invention described herein finds that the overall production of virus particles in the eggs of resistant birds is higher than those in eggs of birds with lower resistance. Also, the eggs of resistant birds have lower embryo mortality than eggs of birds with lower resistance.

The method of the invention can comprise further steps useful in the preparation of a vaccine. For example, the method can further comprise treating the virus to inactivate the virus. Such treatment can be carried out in ovo or can be carried out after removal of the virus from the embryo. The inactivated virus particles removed from the resistant embryo can thus be used to prepare a vaccine to the originally injected virus. Accordingly, the invention can further comprise injecting the vaccine into an animal to effect vaccination against the virus.

Vaccines prepared according to the invention can be used with a variety of animals to effect vaccination against the specific virus. For example, the vaccines can be used in birds and/or mammals. Specific, non-limiting examples of birds that may be vaccinated using a vaccine prepared according to the invention include geese, ducks, turkeys, pigeons, ostriches, and chickens. Specific, non-limiting examples of mammals that may be vaccinated using a vaccine prepared according to the invention include goats, horses, rabbits, rats, mice, pigs, cows, and humans.

In another aspect, the present invention is also directed to a composition for use in a vaccine against a transmittable virus that is pathogenic to a chicken embryo. In certain embodiments, the composition comprises virus particles obtained from a bird embryo of a bird species that exhibits resistance to the transmittable virus (i.e., a “resistant bird species”). In particular embodiments, the resistant bird species is goose or duck. In a specific embodiment, the invention provides a vaccine for an avian influenza virus, the vaccine comprising virus particles obtained from a goose embryo.

In another aspect, the present invention is directed to a method of forming a composition comprising antibodies useful in the therapeutic treatment of a viral disease. The invention thus can provide vaccines for preventing viral infections and therapeutic treatments for patients already infected with a virus.

In one embodiment, a method according to the invention comprises the injecting a live female bird, such as a waterfowl (e.g., goose or duck), with an agent comprising or derived from a viral disease for which a treatment is desired. The agent can comprise a variety of materials capable of eliciting formation of antibodies in the bird. For example, the agent could be active virus. In other embodiments, the agent could a derivative of an active virus, such as a DNA plasmid.

The method according to this aspect of the invention can further comprise providing an incubation period wherein the bird may develop antibodies to the viral disease. The incubation period can vary depending upon the specific virus. The bird is then allowed to lay eggs, which can be retrieved. The eggs laid by the bird should comprise antibodies to the viral disease, and the antibodies in the eggs (i.e., the bird embryos) can be obtained from the eggs and used in the formation of a therapeutic composition. For example, the antibodies could be combined with a pharmaceutically acceptable carrier and/or processed via other known means for producing a therapeutic treatment using antibodies.

The compositions according to this aspect of the invention can be used to treat any of the viral diseases described herein. In one specific embodiment, the viral disease is an avian influenza virus.

The invention also encompasses compositions for therapeutic treatment of a viral disease that are prepared according to the method described above.

### Example 1

### Production of Avian Influenza Virus in Waterfowl Embryos

A stock sample of H3N2 was obtained from ATCC (VR-777) culture collection and used as a viral stock for injection into waterfowl eggs. Two lines, P2SM and JMOP, of goose embryos were used for virus production. Goose embryos at 11 to 17 days of incubation were candled for viability prior to viral injection. Holes were drilled at positions on egg that provided access to either the air sac or chorioallantoic membranes. Approximately 10 to 100 ul of virus stock solution was placed in the air sac or injected into the chorioallantoic membrane using a 26 gauge needle. The hole was sealed using Elmers glue and returned in the upright position into an incubator. The eggs were monitored for viability by candling.

After 3 to 6 days, approximately 0.5-1.0 ml of allantoic fluid were collected from the allantoic cavity of the goose embryos. Samples of the fluid were extracted for RNA and analyzed according to the protocol recommended in the RT-PCR kit (Qiagen) used for detection of H3N2 virus. Briefly, 500 ul of allantoic fluid were mixed with 500 ul of RLT buffer. From this 700 ul was applied to an RNEASY® easy column and microfuged for 15 sec and repeated with remaining sample. 700 ul of Buffer RW1 was applied and the column was microfuged for 15 sec. Next 500 ul of RPE was similarly applied and microfuged and repeated. To elute bound RNA, 30 to 50 ul of RNase free water was added and microfuged for 15 sec and the sample collected for RT-PCR.

RT-PCR was performed using primers for a conserved region of the influenza virus obtained from Integrated DNA Technologies, Inc. (Coralville, Iowa). The primer set included a forward primer, M2F (5′-CAG ATG CAR CGA TTC AGT G-3′), and a reverse primer, M253R (5′-AGG GCA TTT TGG ACA AAG CGT CTA-3′). RT PCR was performed according to the Influenza A virus protocol by Fouchier et al (J. Clin. Microbiology 38, 2000). Briefly, RT-PCR conditions were for 30 min at 42° C. and 4 min at 95° C. followed by 40 cycles of 1 min at 95° C., 1 min at 45° C. and 3 min at 72° C. Approximately 15 ul of nucleotide sample was added to a reaction containing 5 ul of each primer and mixed with RT-PCR buffer containing TAQ enzyme and dNTP. Samples of RT-PCR were analyzed by agarose electrophoresis and ethidium bromide staining.

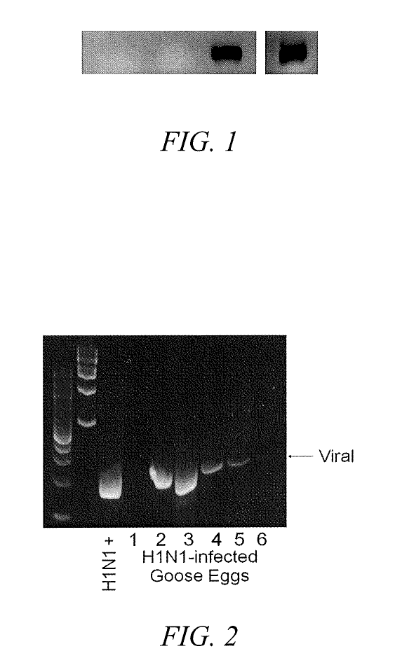

In control eggs (mock injected eggs or eggs injected with virus but harvested after 3 hours), no virus was detected by the RT-PCR. In contrast, H3N2 virus was found to be produced in 8 of 10 of the test goose embryos. Embryos of both goose strains were shown to produce virus. Highest virus production was exhibited upon injection into the allantoic sac compared to the air sac. In FIG. 1 are examples of RT-PCR negative and positive allantoic samples with control. A remarkable feature of goose embryos for the vaccine production of avian influenza virus is the large volume of allantoic fluid harvested from the goose embryo relative to a chicken embryo, up to 10 times the volume. These studies were replicated 7 times utilizing 68 goose eggs demonstrating up to 86% survival and up to 75% of the total eggs displaying significant viral enrichment as determined by RT-PCR of RNA extracts.

### Example 2

### Differential Production of Bird Influenza Virus in Bird Embryos with Differential Viral Resistance

Among the many low pathogenicity strains of influenza virus, there are few reports of unique species-specific susceptibility. However, there are several reports, including a study in northern Europe that report the susceptibility of turkeys to H1N1 (Ludwig, S., Haustein, A., Kaleta, E. F. & Scholtissek, C. (1994). Recent influenza A (H1N1) infections of pigs and turkeys in northern Europe. Virology, 202, 281-286), while there are no known reports of geese susceptible to H1N1 strains. Therefore, to determine if this susceptibility difference between geese and turkeys was also present in the eggs of these two species, goose and turkey eggs were infected with H1N1 (A/Mal/302/54; ATCC VR-98) influenza virus as described in Example 1. The mass of goose compared to turkey eggs was determined to be 2:1, and therefore, standard viral dose used to infect goose eggs, 1×10⁶ virons/egg was adjusted to compensate for mass difference, where 5×10⁵ virions/turkey egg was used. In addition, groups of goose eggs were infected with doses of H1N1 influenza virus both a log below, 1×10⁵ virions/egg, and a log above, 1×10⁷ virons/egg, the normal infectious dose.

Eggs were incubated at 37° C. in a humid chamber and rotated every four hours. Approximately 1.0 mls of allantoic fluid was extracted from both virally infected and sham (PBS) injected eggs five days post infection. RNA was extracted from the allantoic fluid using an RNEASY® kit (Qiagen). Reverse transcriptase-PCR for a conserved region of the matrix gene of the influenza virus was performed on the RNA extract. Significant viral enrichment was demonstrated in goose eggs infected with the H1N1 strain (A/Mal/302/54). A representative gel is shown in FIG. 2. Eggs #2-5 demonstrated significant viral replication, #6 was virus positive although there was not significant replication, and #1 was virus negative;

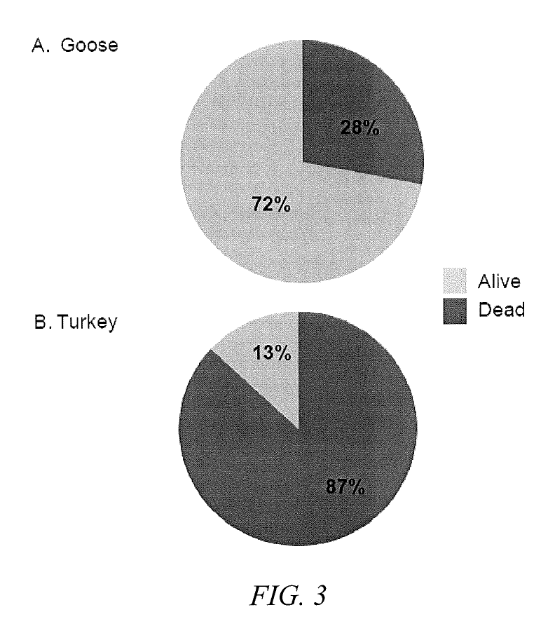

It was demonstrated that there was enhanced resistance in goose eggs infected with the H1N1 relative to turkey eggs (see FIG. 3). Goose eggs were infected with log dilutions of viral titer from 10⁵ to 10⁷, with 13 of 18 eggs remaining viable at day 5 post infection, with no association of death with dose. Turkey eggs were infected with 5×10⁵ virions, the volume compensated standard infectious dose of goose eggs. Seven of the eight turkey eggs infected were dead by day 5 post infection. All sham infected eggs survived. Goose eggs (n=18) and turkey eggs (n=8) were infected with titrated infectious doses of H1N1 ranging from 1×10⁵ to 1×10⁷ virions/egg. Goose eggs were significantly more resistant, irrespective of dose, with 72% remaining alive for 5 days post compared, to 13% of turkey eggs.

These data provide evidence for the first time that bird species exhibiting differences in susceptibility to specific strains of influenza are correlated to the eggs of those strains.

Based on these results it is expected that the resistance of waterfowl birds to high pathogenicity H5N1 may also be expected to be seen with waterfowl eggs, as compared to the susceptibility of live chickens and chicken eggs.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included. Although specific terms are employed herein, they are used in a generic and descriptive sense only and not for purposes of limitation.

1. A method for producing a vaccine against a transmittable virus that is pathogenic to a first bird species susceptible to the virus, said method comprising:
a) injecting an amount of the transmittable virus into the embryo of a second bird species that is different from the first, susceptible bird species and that exhibits resistance to the transmittable virus;
b) incubating the embryo for a period of time after injection effective for virus production in the embryo; and
c) removing fluid from the embryo containing the produced virus.

2. The method of claim 1, further comprising inactivating the virus.

3. The method of claim 2, wherein the virus is inactivated in ovo.

4. The method of claim 2, wherein the virus is inactivated after removal from the embryo.

5. The method of claim 1, wherein the first bird species susceptible to the virus is chicken or turkey.

6. The method of claim 1, wherein the second bird species that exhibits resistance to the transmittable virus is a waterfowl species.

7. The method of claim 6, wherein the second bird species that exhibits resistance to the transmittable virus is goose or duck.

8. The method of claim 1, wherein the transmittable virus injected into the embryo is not a virus that has been genetically reverse engineered to reduce its pathogenicity.

9. The method of claim 1, wherein the transmittable virus is injected in an egg compartment selected from a group consisting of the air sac, the allantoic cavity, and combinations thereof.

10. The method of claim 1, wherein the transmittable viral disease is a native virus or an engineered virus.

11. The method of claim 1, wherein the transmittable virus is selected from the group consisting of West Nile Virus, Hepatitis virus, HIV, RSV, CMV, HSV, ESV, VSV, viral encephalitide, viral hemorrhagic fever, avian influenza virus, and combinations thereof.

12. The method of claim 11, wherein the viral encephalitide are selected from the group consisting of Eastern equine encephalomyelitis virus, Venezuelan equine encephalomyelitis virus, Western equine encephalomyelitis virus, and combinations thereof.

13. The method of claim 11, wherein the avian influenza viruses are selected from the group consisting of H5N1, H5N2, H5N8, H5N9, H7N1, H7N3, H7N4, H7N7, H9N2, and combinations thereof.

14. The method of claim 1, wherein the first bird species is chicken or turkey and wherein the second bird species is goose or duck, and wherein the method further comprises, prior to said injecting step, determining that the transmittable virus is pathogenic to the chicken or turkey and that the goose or duck is resistant to the transmittable virus.

15. A method of protecting an animal against a transmittable virus, the method comprising injecting into the animal a content of a virus produced according to the method of claim 1.

16. The method of claim 5, wherein the animal protected is a bird or mammal.

17. The method of claim 16, wherein the bird is selected from the group consisting of goose, duck, turkey, pigeon, ostrich, and chicken.

18. The method of claim 16, wherein the mammal is selected from the group consisting of goat, horse, rabbit, rat, mice, pig, cow, and human.

19. A method for producing a human vaccine comprising:
a) injecting into a goose embryo an amount of a transmittable virus that is pathogenic to chickens;
b) incubating the goose embryo for a period of time after injection effective for virus production in the goose embryo; and
c) removing fluid from the goose embryo containing the produced virus.

20. A composition for use in a vaccine against a transmittable virus that is pathogenic to a chicken embryo, the composition comprising virus particles from a goose embryo injected with the transmittable virus.
